(19) United States
(12) Patent Application Publication
(10) Pub. No.: US 2005/0065114 A1
(43) Pub. Date: Mar. 24, 2005

(54) Therapeutical treatment with oligo-beta- (1,3) -glucans, drugs used in said treatment

(76) Inventors: Jean-Claude Yvin, Saint Malo (FR); Frank Jamois, La Chapelle Des Fougeretz (FR); Vaclav Vetvicka, Louisville, KY (US)

Correspondence Address: STITES & HARBISON PLLC, 1199 NORTH FAIRFAX STREET, SUITE 900, ALEXANDRIA, VA, 22314, US

(21) Appl. No.: 10/668,665

(22) Filed: Sep. 23, 2003

(51) Int. Cl. A61K 31/715

(52) U.S. Cl. 514/61

(57) ABSTRACT

A therapeutical method comprising administration of a composition comprising an amount of oligo-β-(1,3)-glucan and a pharmaceutically acceptable carrier, to a human being or to a warm-blood animal suffering from a disease selected from the group consisting in a tumor, a cancer, a viral disease, a bacterial disease, a fungal disease, a disease of the immune system, an auto-immune disease or a disease related to a deficiency of immunostimulation, wherein the amount of oligo-β-(1,3)-glucan is effective to treat the disease.

The present invention relates to a therapeutical treatment in which oligo-β-(1,3)-glucans are used, and to drugs used in said treatment.

More particularly, it relates to therapeutical treatments based on the immunostimulant activities of specific oligo-β-(1,3)-glucans.

Glucans which are natural products have been studied extensively and are known as presenting immunostimulating activities. However, it has already been observed that not every compound comprised into naturally occurring glucans are active.

Among the already studied glucans, Laminarin can be cited as presenting immunostimulant activities and consequently as being useful in therapeutical treatments, as disclosed e.g. in the International patent application WO03/045414 in the name of the present inventors.

Laminarin is a natural product extracted from brown algae which presents a molecular weight from about 2500 and 6000 and which consists in a complex mixture of different oligo-and polysaccharides.

Since Laminarin is a mixture of different glucans, even if it presents very interesting therapeutical properties, its use as a drug can be complicated by the difficulty to completely specified the constitutive mixture, in particular, in terms of obtaining corresponding Administrative authorization for marketing.

It was thus necessary to look for other well identified compounds, preferably obtainable by chemical synthesis which present immunostimulant activities.

In the International patent application WO01/57053, the present Assignee has disclosed a chemical process for preparing functionalized β-(1,3)-glucan derivatives.

The functionalized β-(1,3)-glucan derivatives obtained according to said process are not mixtures of compounds but individual compounds presenting a completely identified formula, and could thus be interesting for a pharmaceutical or medical use.

Unexpectedly and surprisingly, the present inventors have found that oligo-β-(1,3)-glucans with 3 to 9 saccharidic units present immunostimulant activities even higher than the immunostimulant activities of Laminarin. In particular, they found that the active oligo-β-(1,3)-glucan not only enhances the phagocytosis but also stimulates NK cells in the mice or the warm-blood animals, and also stimulates the production of TNF-alpha in the mice or the warm-blood animals.

An object of the present invention is thus a therapeutical method comprising administration of a composition comprising an amount of oligo-β-(1,3)-glucan and a pharmaceutically acceptable carrier, to a human being or to a warm-blood animal suffering from a disease selected from the group consisting in a tumor, a cancer, a viral disease, a bacterial disease, a fungal disease, a disease of the immune system, an auto-immune disease or a disease related to a deficiency of immunostimulation, wherein the amount of oligo-β(1,3)-glucan is effective to treat the disease.

Throughout the specification the amount of oligo-β-(1,3)-glucan is considered as “effective” if it allows the obtention of the contemplated medical end such as control or destruction of cancer cells or virally infected cells without producing unacceptable toxic symptoms. Said effective amount will vary with factors such as the particular condition being treated, the physical condition of the patients and the duration of the treatment.

The “pharmaceutical acceptable carrier” is selected from the group comprising pharmaceutically acceptable solvents, suspending agents or vehicles, and in function of the chosen route selected for administration, and keeping in mind standard pharmaceutical practice; “acceptable” means that the carrier is compatible with the other ingredients of the formulation and not injurious to the patient.

More generally, a “pharmaceutically acceptable component” should not present or induce undue adverse side effects such as toxicity, irritation, and allergic response and should be commensurate with a reasonable benefit/risk ratio.

The method according to the invention is particularly useful for the treatment of patients suffering from tumor, viral disease, fungal disease, but also diseases of the immune system, auto-immune diseases, diseases relative to a deficiency of immunostimulating and also cancers, in particular breast cancer, lung cancer, oesophagus cancer, stomach cancer, intestinal cancer or colon cancer.

More particularly, the present inventors have found that the active oligo-β(1,3)-glucans are those which present the following formula (1): 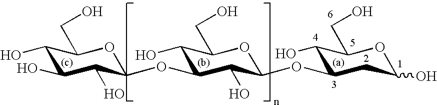
in which n=1 to 7,preferably n=2 or 3, and pharmaceutically acceptable salts thereof.

Advantageously, the active oligo-β(1,3)-glucans are those of formula (1) above,
in which n=2, i.e. β-D-glucopyranosyl-(1→3)-β-D-glucopyranosyl-(1→3)-β-D-glucopyranosyl-(1→3)-β-D-glucopyranose, which is called Laminaritetraose, or in which n=3, i.e. the β-D-glucopyranosyl-(1→3)-β-D-glucopyranosyl-(1→3)-β-D-glucopyranosyl-(1→3)-β-D-glucopyranosyl glucopyranosyl-(1→3)-β-D-glucopyranose, which is called Laminaripentaose.

Those compounds can be synthesized by de-protection and purification of the compounds prepared according to the process disclosed in WO01/57053. The method for de-protection and purification can be the one described with reference to D-Laminaribiose in FR2777281

In the method according to the invention, the composition comprising active oligo-β(1,3)-glucan and pharmaceutically acceptable carrier is administered intravenously, intraperitoneally, or orally to the patient.

It can also be presented as a bolus, an electuary, or a paste.

According to another object of the invention, the method according to the invention further comprises administration of a chemotherapeutical agent, or of a potentiator.

The term “potentiator” designates a material that improves or increases the efficiency of oligo-β-(1,3)-glucan or acts on the immune system as immuno-modulator.

When oligo-β-(1,3)-glucan is combined with chemotherapeutic agents, or potentiators, then the therapy can be called a “combination therapy”.

Combination therapy can be sequential, which means that the treatment is carried out with one agent first and then with the second agent; or it can be a treatment with both agents at the same time.

The sequential therapy can be performed within a reasonable time after the completion of the first therapy before beginning the second one. The treatment with both agents at the same time can be in the same daily dose or separate doses.

For example:
in the case of retroviral infection, a combination therapy may consist in treatment with an oligo-β-(1,3)-glucan together with nucleosides analogues, (with inhibitors of reverse transcriptase) , such as AZT or with proteases inhibitors such as Ritonavir.
in the case of cancer diseases a combination therapy may consist in treatment with an oligo-β-(1,3)-glucan together with topo-isomerase inhibitors, such as Topotecam, Antracycline, or antimetabolites such a Cytarabine, Fluorouracil and others.

The present invention also relates to therapeutical composition under the form of spray, aerosol, tablet, capsule, injection, ointment, pulmonary aerosol comprising a therapeutically effective amount of oligo-1-3-β-glucan of formula (1): 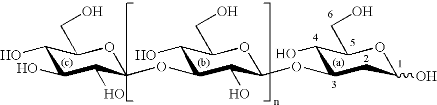
in which n=1 to 7, preferably n=2 or n=3, or a salt pharmaceutically acceptable salt thereof, and a pharmaceutical acceptable carrier, said composition being free of any other glucan.

Oral formulations suitable for use in connection with the practice of the present invention include capsules, gels, cachets, tablets, effervescent or non-effervescent powders, tablets, or granules; they may consist of a solution, of a suspension in an aqueous or non-aqueous liquid, of an oil-in-water liquid emulsion or of a water-in-oil emulsion.

Generally, the said formulations may be prepared by uniformly mixing the active ingredient, i.e. especially soluble oligo-β-(1,3)-glucan with liquid carriers or finely divided solid carriers or both, and then if necessary by shaping the product.

Suitable solid carriers comprise lactose, sucrose, gelatin, agar and bulk powders.

Suitable liquid carriers include water, pharmaceutically acceptable fats and oils, alcohols or other organic solvents, including esters, emulsions, syrups or elixirs, solutions and/or suspensions, and solutions and/or suspensions reconstituted from non-effervescent granules and effervescent preparations reconstituted from effervescent granules.

They also may contain, for example, preservatives, emulsifying agents, suspending agents, diluents, sweeteners, thickeners, and melting agents; preferred liquid carriers are edible oils, for example, corn or canola oils, as well as, polyethylene glycols (PEG).

The therapeutical forms, intended for oral administration, may comprise a non-toxic, pharmaceutically acceptable, inert carrier selected from the group comprising lactose, starch, sucrose, glucose, methyl cellulose, magnesium stearate, dicalcium phosphate, calcium sulfate, mannitol, sorbitol, cyclodextrin, and cyclodextrin derivatives, or the like.

Capsules or tablets containing an oligo-β-(1,3)-glucan according to the invention should preferably be easily formulated and made easy to swallow or to chew. Tablets may contain suitable carriers, binders, lubricants, diluents, disintegrating agents, coloring agents, flavoring agents, flow-inducing agents, or melting agents. A tablet may be produced by compression or molding, optionally with one or more classical additional ingredients.

Compressed tablets may be prepared by compressing the active ingredient in a free flowing form (e.g., powder, granules) optionally mixed with a binder (e.g., gelatin, hydroxypropylmethylcellulose), lubricant, inert diluent, preservative, disintegrant (e.g., sodium starch glycolate starch, gelatin, natural sugars such as glucose or beta-lactose, corn sweeteners, natural and synthetic gums such as, sodium alginate, carboxymethylcellulose, polyethylene glycol, waxes, or the like. Lubricants used in these dosage forms include sodium oleate, sodium stearate, magnesium stearate, sodium acetate, sodium chloride, or the like. Disintegrating agents include, for example, starch, methyl cellulose, agar, bentonite, xanthan gum or the like. Molded tablets are made by molding in a suitable machine a mixture of the powdered active ingredient moistened with an inert liquid diluent.

The tablets are optionally coated and may be formulated so as to provide slow-or controlled-release of the active ingredient. Tablets may also optionally be provided with an enteric coating to provide release in parts of the gut other than the stomach.

The following examples are intended to illustrate the invention in particular, to illustrate the activity of oligo-β-(1,3)-glucan derivatives.

### EXAMPLES

In the examples, the following abbreviations are used:
APTS,H₂0: p-toluenesulfonic acid monohydrate
CSA: Camphorsulfonic acid
DDQ: 2,3-dichloro-5,6-dicyano-1,4-benzoquinone
NIS: N-Iodosuccinimide
Sn(OTf)₂: tin trifluoromethanesulfonate

### Example 1

### Preparation of Laminaritetraose

a) To a solution of 2.88 g (1.89 mmol) of benzyl 2-O-benzoyl-4,6-O-benzylidene-β-D-glucopyroanosyl-(1→3)-2-O-benzoyl-4,6-O-benzylidene-β-D-glucopyranosyl-(1→3)-2-O-benzoyl-4,6-O-benzylidene-β-D-glucopyranosyl-(1→3)-2-O-benzoyl-4,6-O-benzylidene-β-D-glucopyranoside of formula 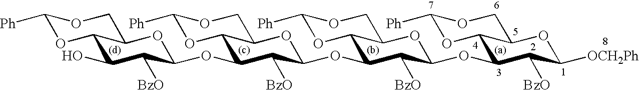
obtained according to example 11 of WO01/57053, in 84 mL of a acetone/methanol/water (1:4:1) mixture, 0.439 g (1.89 mmol) of CSA is added and the mixture is heated under stirring at 70° C. for 3 hours. The solution is then cooled to room temperature in an ice bath, neutralized with triethylamine and then concentrated. The crude product is purified by chromatography (E. Merck 60H 5-40 μm Silica Gel), eluting with toluene/ethyl acetate (9:1), to give benzyl 2-O-benzoyl-β-D-glucopyranosyl-(1→3)-2-O-benzoyl-β-D-glucopyranosyl-(1→3)-2-O-benzoyl-β-D-glucopyranosyl-(1→3)-2-O-benzoyl-β-D-glucopyranoside of formula 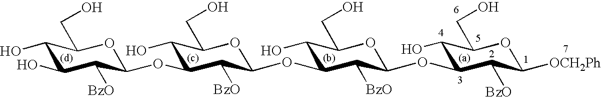
TLC: R_{f}=0.4 (dichloromethane/methanol, 17:3)
m.p. (0° C.)=152-154
[α]²⁰_{D}=+20 (c=1, 0; methanol)
NMR ¹³C (MeOD, 101 MHz) δ (ppm): 167.06, 166.12, 165.98, 165.79 (C═O); 138.43 (C ipso of Bn); 134.26, 134.20, 134.11, 133.88 (C ipso of Bz); 130.82-128.56 (C arom.); 102.22 (C1b or C1c); 101.81, 101.74 (C1d et C1b or C1c); 100.91 (C1a); 83.92 (C3a) ; 82.77, 82.42 (C3b, C3c); 78.31, 77.95, 77.75 (2C) (C5a, C5b, C5c, C5d); 76.05 (C3d); 75.24 (C2d); 74.56, 74.45, 74.34 (C2a, C2b, C2c) ; 71.49 (C4d); 71.38 (C7); 70.02, 69.79, 69.76 (C4a, C4b, C4c) ; 62.53, 62.42, 62.36 (2C) (C6a, C6b, C6c, C6d).

NMR ¹H (MeOD, 400 MHz) δ (ppm): 7.50-6.88 (m, 25H, H arom.); 4,79 (d, 1H, H2a, J_{H2a-H1a}=J_{H2a-H3a}=8.7 Hz); 4.74 (t, 1H, H2b ou H2c, J_{H2-H1}=J_{H2-H3}=8.8 Hz); 4.72 (H2b or H2c, J_{H2-H1}=J_{H2-H3}=8.7 Hz); 4.71 (t, 1H, H2d, J_{H2d-H1d}=J_{H2d-H3d}=8.6 Hz); 4.62 (d, 1H, H7, J_{H7-H7′}=12.5 Hz); 4.57 (d, 1H, H1b or H1c, J_{H1-H2}=8.0 Hz); 4.47 (d, 1H, H1b or H1c, J_{H1-H2}=8.0 Hz); 4.41 (d, 1H, H7′, J_{H7′-H7}=12.0 Hz); 4.40 (d, 1H, H1a, J_{H1a-H2a}=8.4 Hz); 4.37 (d, 1H, H1d, J_{H1d-H2d}=8.1 Hz); 3.82-3.69 (m, 6H, H3a, H3b or H3c, 4 H6); 3.62-3.52 (m, 5H, H3b or H3c, 4 H6); 3.38-3.05 (m, 9H, H3d, H4a, H4b, H4c, H4d, H5a, H5b, H5c, H5d). MS:
[M+Na]⁺ m/z: calculated=1195, 3634; found=1195, 3627
[M+K]⁺ m/z: calculated=1211, 3374; found=1211, 3461

b) After dissolution of 2.29 g (1.96 mmol) of 2-O-benzoyl-β-D-glucopyranosyl-(1→3)-2-O-benzoyl-β-D-glucopyranosyl-(1→3) -2-O-benzoyl-β-D-glucopyranosyl-(1→3) -2-O-benzoyl-β-D-glucopyranoside in 70 mL of anhydrous methanol, 67 mg (2.91 mmol) of sodium are added and the solution is heated to 50° C. After stirring during 8 hours, the solution is cooled to room temperature, the reaction mixture is neutralized with acetic acid and concentrated. Then, a suspension of the crude product in water is prepared and methyl benzoate formed during the reaction is removed by extractions with dichloromethane. After co-evaporation of the resulting aqueous layer with absolute ethanol, the desired product is purified by gel permeation using Sephadex G-15 and water as eluent. The purified fractions are then freeze-dried to provide benzyl β-D-glucopyranosyl-(1 →3)-β-D-glucopyranosyl-(1→3)-β-D-glucopyranosyl-(1→3) -β-D-glucopyranoside of formula

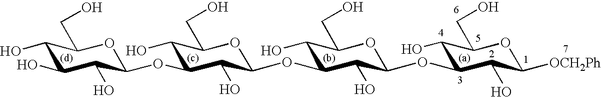

TLC: R_{f}=0.5 (ethyl acetate/isopropanol/water, 3:3:1)

[α]²⁰_{D}=−29 (c=1, 0; water)

NMR ¹³C (D₂O, 101 MHz) δ (ppm): 136.74 (C ipso Ph); 129.05, 129.01, 128.79 (C arom.); 103.08, 102.81, 102.78 (C1b, C1c, C1d); 101.20 (C1a); 84.53, 84.40, 84.17 (C3a, C3b, C3c); 76.26, 75.87 (3C), 75.79 (C3d, C5a, C5b, C5c, C5d); 73.70, 73.57, 73.52, 73.21 (C2a, C2b, C2c, C2d); 71.80 (C7); 69.83 (C4d), 68.42, 68.36, 68.33 (C4a, C4b, C4c); 60.92 (C6a, C6b, C6c, C6d).

NMR ¹H (D₂O, 400 MHz) δ (ppm): 7.35-7.26 (m, 5H, H arom.); 4.81 (d, 1H, H7, J_{H7-H7′}=11.6 Hz); 4.65 (d, 1H, H1b, H1c or H1d, J_{H1-H2}=8.2 Hz); 4.62 (d, 1H, H1b, H1c or H1d, J_{H1-H2}=8.6 Hz); 4.62 (d, 1H, H7′, J_{H7′-H7}=11.4 Hz); 4.61 (d, 1H, H1b, H1c or H1d, J_{H1-H2}=8.0 Hz); 4.42 (d, 1H, H1a, J_{H1a-H2a}=8.0 Hz); 3.81-3.76 (m, 4H, H6); 3.66-3.56 (m, 7H, H3a, H3b, H3c, 3 H6); 3.43-3.19 (m, 13H, H2a, H2b, H2c, H2d, H3d, H4a, H4b, H4c, H4d, H5a, H5b, H5c, H5d). MS:
[M+Na]⁺m/z: calculated=779, 2586; found=779, 2580
[M+K]⁺m/z : calculated=795, 2325; found=795, 2380

c) 448 mg (0.10 mmol) of palladium acetate are added to a solution of 1.12 g (1.48 mmol) of benzyl β-D-glucopyranosyl-(1→3)-β-D-glucopyranosyl-β(1→3)-β-D-glucopyranosyl-(1→3)-β-D-glucopyranoside in 20 mL of a methanol/water (1:1) mixture, and the suspension is then stirred vigorously during 5 hours under hydrogen atmosphere at room temperature. After filtration and concentration, the product is submitted to gel permeation purification (Sephadex G-15, water as eluent) to give, after freeze-drying of the purified fractions, 969 mg of the desired D-glucopyranosyl-(1→3)-β-D-glucopyranosyl-(1→3)-β-D-glucopyranosyl-(1→3)-β-D-glucopyranose of formula

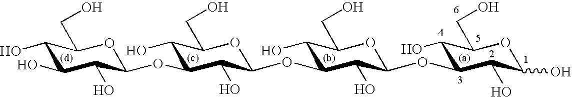

Overall yield (3 steps)=69%

TLC: R_{f}=0.2 (ethyl acetate/isopropanol/water, 3:3:2)

[α]²⁰_{D}=−5 (t=10 min, c=1, 0; water)

NMR ¹³C (D₂O, 101 MHz) δ (ppm): 103.11 (2C), 102.95, 102.85, 102.82 (2C) (2 C1b, 2 C1c, 2 C1d); 95.98 (C1aβ); 92.32 (C1aα); 84.71, 84.46, 84.33, 84.29, 82.51 (2 C3a, 2 C3b, 2C3c); 76.29, 75.90, 75.83, 74.14, 73.74, 73.61, 73.56, 71.51, 71.37, 69.87, 68.40, 68.36; 60.98, 60.81 (8 C6). NMR ¹H (D₂O, 400 MHz) δ (ppm): 5.13 (d, 1H, H1aα, J_{H1aα-H2a}=3.7 Hz); 4.67 (d, 3H, H1, J_{H1-H2}=8.9 Hz); 4.65 (d, 3H, H1, J_{H1-H2}=7.8 Hz); 4.57 (d, 1H, H1aβ, J_{H1aβ-H2a}=8.0 Hz); 3.83-3.59 (m, 24H); 3.47-3.23 (m, 24H). MS:
[M+Na]⁺m/z: calculated=689, 2116; found=689, 2121
[M+K]⁺m/z: calculated=705, 1856; found=705, 1843

### Example 2

### Preparation of Laminaripentaose

a) NIS (1.48 g, 6.6 mmol) and Sn(OTf)₂ (230 mg, 0.6 mmol) are added to a solution of 3.37 g (6.1 mmol) of ethyl 2-O-benzoyl-4,6-O-benzylidene-3-O-(2-methylnaphtyl)-1-thio-β-D-glucopyranoside of formula 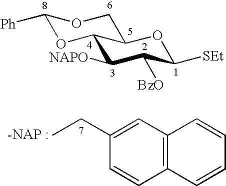
obtained according to example 4 of WO01/57053 and 8.38 g (5.5 mmol) of benzyl 2-O-benzoyl-4,6-O-benzylidene-β-D-glucopyranosyl-(1→3) -2-O-benzoyl-4,6-O-benzylidene-β-D-glucopyranosyl-(1→3) -2-O-benzoyl-4,6-O-benzylidene-β-D-glucopyranosyl-(1→3) -2-O-benzoyl-4,6-O-benzylidene-β-D-glucopyranoside of formula 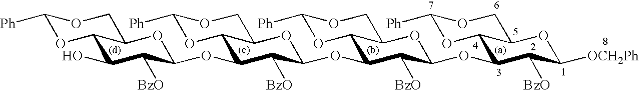
obtained according to example 11 of WO01/57053, in anhydrous dichloromethane (180 mL) in the presence of 4-A molecular sieves (1.8 g) . The reaction mixture is stirred at 0° C. for 2 hours and then quenched by adding triethylamine. After filtration through a bed of Celite, the resulting solution is concentrated and the crude product is purified by chromatography (E. Merck 60H 5-40 μm Silica Gel), eluting with toluene/ethyl acetate (93:7, 9:1 and 17:3), to provide 8.20 g of benzyl 2-O-benzoyl-4,6-O-benzylidene-3-O-(2-methylnaphtyl)-β-D-glucopyranosyl-(1→3)-2-O-benzoyl-4,6-O-benzylidene-β-D-glucopyranosyl-(1→3)-2-O-benzoyl-4,6-O-benzylidene-β-D-glucopyranosyl-(1→3)-2-O-benzoyl-4,6-O-benzylidene-β-D-glucopyranosyl-(1→3)-2-O-benzoyl-4,6-O-benzylidene-β-D-glucopyranoside of formula

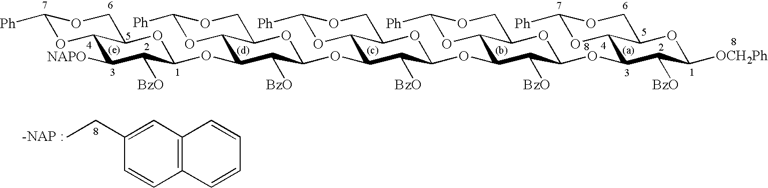

Yield: 74%

TLC: R_{f}=0.6 (toluene/ethyl acetate, 8:2)

m.p. (° C.)=162-165

[α]²⁰_{D}=+26 (c=1,0; dichloromethane)

NMR ¹³C (CDCl₃, 100 MHz) δ (ppm): 165.05, 164.76, 164.72, 164.60, 164.51 (5 C=O); 137.35 (2C), 137.26 (2C), 137.24, 136.65, 135.44, 133.59, 133.50, 133.42, 133.13 (2C), 133.03, 132.81 (14 C ipso); 129.79-125.31 (C arom.); 102.00, 101.33, 101.14, 100.90, 100.62 (C7a, C7b, C7c, C7d, C7e); 99.43 (C1a); 98.64 (C1e); 98.08 (C1b*); 97.19 (C1c*); 96.78 (C1d*); 81.30 (C4e); 78.71 (C4a); 78.25 (C3e); 78.02 (C4c*); 77.91 (C4d*); 77.30 (C4b*); 76.57 (C3c*); 74.47 (2C) (C3a, C3b*); 74.09 (2C) (C2a, C3d*); 73.77 (C8e); 73.50 (C2d*); 73.29 (C2e); 72.85 (C2b*); 72.35 (C2c*); 70.21 (C8a); 68.70 (3C), 68.63 (2C), 68.54 (C6a, C6b, C6c, C6d, C6e); 66.40, 66.06, 65.61, 65.43, 65.41 (C5a, C5b, C5c, C5d, C5e).

NMR ¹H (CDCl₃, 400 MHz) 6 (ppm): 7.85-7.04 (m, 62H, H arom.); 5.51 (s, 1H, H7); 5.45 (s, 1H, H7); 5.36 (t, 1H, H2e, J_{H2e-H1e}=J_{H2e-H3e}=7.8 Hz); 5.14 (t, 1H, H2c*, J_{H2c-H1c}=J_{H2c-H3c}=4.7 Hz); 5.04 (d, 1H, H1e, J_{H1e-H2e}=7.5 Hz); 4.96 (t, 1H, H2a, J_{H2a-H1a}=J_{H2a-H3a}=8.4 Hz); 4.94 (d, 1H, H1c*, J_{H1c-H2c}=5.2 Hz); 4.92 (s, 1H, H7); 4.91 (d, 1H, H8e, J_{H8e-H8′e}=13.0 Hz); 4.89 (d, 1H, H1d*, J_{H1d-H2d}=7.3 Hz); 4.83 (t, 1H, H2b*, J_{H2b-H1b}=J_{H2b-H3b}=4.3 Hz); 4.81 (t, 1H, H2d*, J_{H2d-H1d}=J_{H2d-H3d}=5.9 Hz); 4.80 (d, 1H, H8′e, J_{H8′e-H8e}=11.3 Hz); 4.79 (d, 1H, H1b*, J_{H1b-H2b}=5.9 Hz); 4.75 (d, 1H, H8a, J_{H8a-H8′a}=12.6 Hz); 4.72 (s, 1H, H7); 4.69 (s, 1H, H7); 4.49 (d, 1H, H8′a, J_{H8a-H8a}=12.6 Hz); 4.46 (d, 1H, H1a, J_{H1a-H2a}=7.8 Hz); 4.33 (dd, 1H, H6a, J_{H6a-H5a}=4.6 Hz, J_{H6a-H6′a}=10.2 Hz); 4.21 (dd, 1H, H6, J_{H6-H5}=4.8 Hz, J_{H6-H6′}=10.4 Hz); 4.14-4.12 (m, 2H, H6); 4.09-3.99 (m, 2H, H3d*, H6); 4.06 (t, 1H, H3a, J_{H3a-H2a}=J_{H3a-H4a}=9.0 Hz); 4.01 (t, 1H, H3c*, J_{H3c-H2c}=J_{H3c-H4c}=10.4 Hz); 3.99 (t, 1H, H4c*, J_{H4c-H3c}=J_{H4c-H5c}=8.5 Hz); 3.91-3.86 (m, 1H, H3b*); 3.89 (t, 1H, H4e, J_{H4e-H3e}=J_{H4e-H5e}=8.3 Hz); 3.86 (t, 1H, H3e, J_{H3e-H2e}=J_{H3e-H4e}=8.5 Hz); 3.72 (t, 1H, H6′, J_{H6′-H5}=J_{H6′-H6}=10.8 Hz); 3.69 (t, 1H, H6′a, J_{H6′a-H5a}=J_{H6a-H6a}=10.7 Hz); 3.67 (t, 1H, H4b*, J_{H4b-H3b}=J_{H4b-H5b}=8.4 Hz); 3.59-3.36 (m, 8H, H5a, H5b, H5c, H5d, H5e, 3 H6); 3.24 (t, 1H, H4d*, J_{H4d-H3d}=J_{H4d-H5d}=8.6 Hz); 3.20 (t, 1H, H4a, J_{H4a-H3a}=J_{H4a-H5a}=9.3 Hz).

*: units can be inverted MS:

[M+Na]⁺ m/z: calculated=2041, 6616; found=2041, 6687 Microanalyse (C₂₈H₃₈O₁₉):

| | | |
|---|---|---|
| Calculated | C = 70.16% | H = 5.29% |
| Found | C = 70.23% | H = 5.30% |

b) To a solution (2 g, 0.99 mmol) of benzyl 2-O-benzoyl-4,6-O-benzylidene-3-O-(2-methylnaphtyl)-β-D-glucopyranosyl-(1→3)-2-O-benzoyl-4,6-O-benzylidene-β-D-glucopyranosyl-(1→3)-2-O-benzoyl-4,6-O-benzylidene-β-D-glucopyranosyl-(1→3)-2-O-benzoyl-4,6-O-benzylidene-β-D-glucopyranosyl-(1→3)-2-O-benzoyl-4,6-O-benzylidene-β-D-glucopyranoside in 20 mL of a dichloromethane/methanol (4:1) mixture, 675 mg (2.97 mmol) of DDQ are added and the suspension is stirred for 5 hours at room temperature. Then, the mixture is diluted with dichloromethane, washed with aqueous sodium bicarbonate solution (5%), with water, dried with magnesium sulfate and concentrated. Purification by chromatography (E. Merck 60H 5-40 μm Silica Gel) with a toluene/ethyl acetate (17:3 and 8:2) eluent provides 1.60 g of the desired benzyl 2-O-benzoyl-4,6-O-benzylidene-β-D-glucopyranosyl-(1→3)-2-O-benzoyl-4,6-O-benzylidene-β-D-glucopyranosyl-(1→3)-2-O-benzoyl-4,6-O-benzylidene-β-D-glucopyranosyl-(1→3)-2-O-benzoyl-4,6-O-benzylidene-β-D-glucopyranosyl-(1→3)-2-O-benzoyl-4,6-O-benzylidene-β-D-glucopyranoside of formula

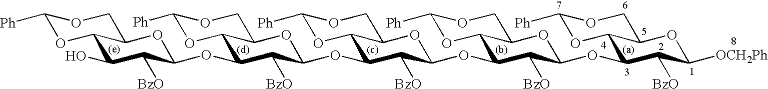

Yield: 86%

TLC: R_{f}=0.4 (toluene/ethyl acetate, 8:2)

m.p. (° C.)=176-179

[α]²⁰_{D}=+3 (c=1, 0; dichloromethane)

NMR ¹³C (CDCl₃, 100 MHz) δ (ppm): 165.86, 164.84, 164.73, 164.67, 164.54 (5 C═O); 137.32, 137.21 (3C), 137.01, 136.65, 133.83, 133.56, 133.52, 133.15 (2C) (11 C ipso); 129.93-125.32 (C arom.); 102.02, 101.76, 101.37, 100.93, 100.62 (C7a, C7b, C7c, C7d, C7e); 99.41 (C1a); 98.40 (C1e); 98.09 (C1b*); 97.23 (C1c*); 96.84 (C1d*); 80.81 (C4e); 78.72 (C4a); 77.99 (2C) (C4c*, C4d*); 77.30 (C4b*); 76.57 (C3c*); 74.71 (C2e); 74.52 (2C) (C3a, C3b*); 74.29 (C3d*) 74.09 (C2a); 73.44 (C2d*); 72.89 (C2b*); 72.48 (C3e); 72.35 (C2c*); 70.21 (C8a); 68.69 (2C), 68.63 (2C), 68.53 (C6a, C6b, C6c, C6d, C6e); 66.40, 66.03, 65.61, 65.41 (2C) (C5a, C5b, C5c, C5d, C5e).

NMR ¹H (CDCl₃, 400 MHz) δ (ppm): 7.96-7.04 (m, 55H, H arom. ); 5.51 (s, 1H, H7); 5.42 (s, 1H, H7); 5.20 (t, 1H, H2e, J_{H2e-H1e}=J_{H2e-H3e}=10.4 Hz); 5.18 (t, 1H, H2c*, J_{H2c-H1c}=J_{H2c-H3c}=5.2 Hz); 5.09 (d, 1H, H1e, J_{H1e-H2e}=7.6 Hz); 4.97 (d, 1H, H1c*, J_{H1c-H2c}=4.9 Hz); 4.94 (t, 1H, H2a, J_{H2a-H1a}=J_{H2a-H3a}=8.4 Hz); 4.90 (m, 2H, H1d*, H2d*); 4.90 (s, 1H, H7); 4.84 (t, 1H, H2b*, J_{H2b-H1b}=J_{H2b-H3b}=4 . 5 Hz); 4.80 (d, 1H, H1b*, J_{H1b-H2b}=4.6 Hz); 4.76 (d, 1H, H8a, J_{H8a-H8′a}=13.1 Hz); 4.73 (s, 1H, H7); 4.68 (s, 1H, H7); 4.50 (d, 1H, H8′a, J_{H8′a-H8a}=12.6 Hz); 4.46 (d, 1H, H1a, J_{H1a-H2a}=7.8 Hz); 4.33 (dd, 1H, H6, J_{H6-H5}=4.7 Hz, J_{H6-H6′}=10.3 Hz); 4.21 (dd, 1H, H6, J_{H6-H5}=4.9 Hz, J_{H6-H6′}=10.4 Hz); 4.13-3.88 (m, 9H, H3a, H3b, H3c, H3d, H3e, H4c*, 3 H6); 3.67 (t, 1H, H4c*, J_{H4c-H3c}=J_{H4c-H5c}=8.7 Hz); 3.65 (t, 1H, H4e, J_{H4e-H3e}=J_{H4e-H5e}=9.3 Hz); 3.72-3.34 (m, 10H, H5a, H5b, HSc, H5d, H5e, 5 H6); 3.23 (t, 1H, H4d*, J_{H4d-H3d}=J_{H4d-H5d}=10.0 Hz); 3.20 (t, 1H, H4a, J_{H4a-H3a}=J_{H4a-H5a}=9.5 Hz); 2.69 (d, 1H, OH, J_{OH-H3d}=3.4 Hz) .

*: uni ts can be inverted MS:

[M+Na]⁺ m/z: calculated=1901, 5990; found=1901, 6028 Microanalysis (C₂₈H₃₈O₁₉):

| | | |
|---|---|---|
| Calculated | C = 68.36% | H = 5.25% |
| Found | C = 68.05% | H = 5.25% |

c) To a solution of 2.10 g (1.12 mmol) of benzyl 2-O-benzoyl-4,6-O-benzylidene-β-D-glucopyranosyl-(1→3)-2-O-benzoyl-4,6-O-benzylidene-β-D-glucopyranosyl-(1→3)-2-O-benzoyl-4,6-O-benzylidene-β-D-glucopyranosyl-(1→3)-2-O-benzoyl-4,6-O-benzylidene-β-D-glucopyranosyl-(1→3)-2-O-benzoyl-4,6-O-benzylidene-β-D-glucopyranoside in 60 mL of a acetone/methanol/water (1:4:1) mixture, 0.213 g (1.12 mmol) of APTS,H₂O is added and the mixture is heated under stirring at 70° C. for 3 hours. The solution is then cooled to room temperature in an ice bath, neutralized with triethylamine and then concentrated. The crude product is purified by chromatography (E. Merck 60H 5-40 μm Silica Gel), eluting with toluene/ethyl acetate (9:1), to give benzyl 2-O-benzoyl-β-D-glucopyranosyl-(1→3)-2-O-benzoyl-β-D-glucopyranosyl-(1→3)-2-O-benzoyl-β-D-glucopyranosyl-(1→3)-2-O-benzoyl-β-D-glucopyranosyl-(1→3)-2-O-benzoyl-β-D-glucopyranoside of formula

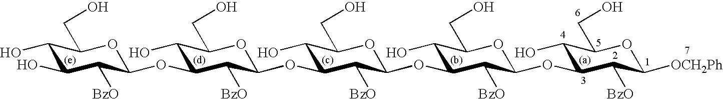

TLC: R_{f}=0.3 (dichloromethane/methanol, 17:3)

m.p. (° C.)=171-174

[α]²⁰_{D}=+21 (c=1,0; methanol)

NMR 13C (MeOD, 101 MHz) 6 (ppm): 167.06, 166.11, 165.94, 165.82, 165.78 (C═O); 138.43 (C ipso of Bn); 134.26, 134.25, 134.18, 134.13, 133.87 (C ipso of Bz); 130.84-128.55 (C arom.); 102.21, 101.82, 101.76, 101.75 (C1b, C1c, C1d, C1e); 100.87 (C1a); 83.91 (C3a); 82.79, 82.37, 82.27 (C3b, C3c, C3d); 78.36, 78.09, 77.94, 77.77 (2C) (C5a, C5b, C5c, C5d, C5e); 76.06 (C3e); 75.21 (C2e); 74.56, 74.44, 74.35 (2C) (C2a, C2b, C2c, C2d); 71.49 (C4e); 71.35 (C7); 70.01, 69.78, 69.70, 69.63 (C4a, C4b, C4c, C4d); 62.56, 62.45 (2C), 62.35 (2C) (C6a, C6b, C6c, C6d, C6e).

NMR ¹H (MeOD, 400 MHz) 6 (ppm): 7.48-6.87 (m, 30H, H arom.); 4.79-4.52 (m, 5H, H2a, H2b, H2c, H2d, H2e); 4.60 (d, 1H, H7, J_{H7-H′}=12.5 Hz); 4.53 (d, 1H, H1b, H1c, H1d or H1e, J_{H1-H2}=8.0 Hz); 4.39 (d, 1H, H1b, H1c, H1d or H1e, J_{H1-H2}=7.8 Hz); 4.39 (d, 1H, H7′, J_{H7′-H7}=12.2 Hz); 4.38 (d, 1H, H1b, H1a, J_{H1a-H2a}=7.8 Hz); 4.31 (d, 1H, H1b, H1c, H1d or H1e, J_{H1-H2}=8.2 Hz); 4.29 (d, 1H, H1b, H1c, H1d or H1e, J_{H1-H2}=8.3 Hz); 3.79-3.72 (m, 6H, 2 H3, 4 H6); 3.67-3.43 (m, 8H, 2 H3, 6 H6); 3.36-3.02 (m, 9H, H3e, H4a, H4b, H4c, H4d, H4e, H5a, H5b, H5c, H5d, H5e). MS:
[M+Na]⁺ m/z: calculated=1461, 4425; foun =1461, 4413
(M+K]⁺ m/z: calculated=1477, 4164; found=1477, 4237
[M−H+2Na]⁺ m/z: calculated=1483, 4244; found=1483, 4241

d) After dissolution of 1.62 g (1.13 mmol) of benzyl 2-O-benzoyl-β-D-glucopyranosyl-(1→3)-2-O-benzoyl-β-D-glucopyranosyl-(1→3)-2-O-benzoyl-β-D-glucopyranosyl-(1→3)-2-O-benzoyl-β-D-glucopyranosyl-(1→3) -2-O-benzoyl-β-D-glucopyranoside in 54 mL of anhydrous methanol, 39 mg (1.70 mmol) of sodium are added and the solution is heated to 50° C. After stirring during 7 hours, the solution is cooled to room temperature, the reaction mixture is neutralized with acetic acid and concentrated. Then, a suspension of the crude product in water is prepared and methyl benzoate formed during the reaction is removed by extractions with dichloromethane. After co-evaporation of the resulting aqueous layer with absolute ethanol, the desired product is purified by gel permeation using Sephadex G-15 and water as eluent. The purified fractions are then freeze-dried to provide benzyl β-D-glucopyranosyl-(1→3)-13-D-glucopyranosyl-(1→3)-β-D-glucopyranosyl-(1→3)-β-D-glucopyranosyl-(1→3)-β-D-glucopyranoside of formula

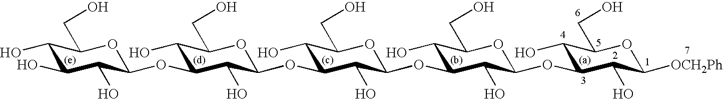

TLC: R_{f}=0.4 (ethyl acetate/isopropanol/water, 3:3:1) [α]²⁰_{D}=−21 (c=1,0; water)

NMR ¹³C (D₂O, 101 MHz) δ (ppm): 136.74 (C quat. arom.); 129.05, 129.02, 128.79 (C arom.); 103.08, 102.80 (3C) (C1b, C1c, C1d, C1e); 101.20 (C1a); 84.53, 84.40, 84.20 (2C) (C3a, C3b, C3c, C3d); 76.26, 75.87 (4C), 75.79 (C3e, C5a, C5b, C5c, C5d, C5e); 73.70, 73.57 (2C), 73.53, 73.21 (C2a, C2b, C2c, C2d, C2e); 71.81 (C7); 69.83 (C4e), 68.42, 68.36, 68.33 (2C) (C4a, C4b, C4c, C4d); 60.92 (C6a, C6b, C6c, C6d, C6e).

NMR ¹H (D₂0, 400 MHz) 6 (ppm): 7.35-7.28 (m, 5H, H arom.); 4.81 (d, 1H, H7, J_{H7-H7′}=11.6 Hz); 4.66 (d, 1H, H1b, H1c, H1d or H1e, J_{H1-H2}=8.0 Hz); 4.65 (d, 1H, H1b, H1c, H1d or H1e, J_{H1-H2}=8.4 Hz); 4.63 (d, 1H, H1b, H1c, H1d or H1e, J_{H1-H2}=9.0 Hz); 4.63 (d, 1H, H7′, J_{H7′-H7}=12.1 Hz); 4.61 (d, 1H, H1b, H1c, H1d or H1e, J_{H1-H2}=7.4 Hz); 4.41 (d, 1H, H1a, J_{H1a-H2a}=8.0 Hz); 3.81-3.76 (m, 5H, H6); 3.66-3.56 (m, 9H, H3a, H3b, H3c, H3d, 4 H6); 3,43-3,20 (m, 16H, H2a, H2b, H2c, H2d, H2e, H3e, H4a, H4b, H4c, H4d, H4e, H5a, H5b, H5c, H5d, H5e). MS:
[M+Na]⁺ m/z: calculated=941, 3114; found=941, 3114
[M+K]⁺ m/z: calculated=957, 2853; found=957, 2821
[M−H+2Na]⁺ m/z: calculated=963, 2934; found=963, 2939

e) 192 mg (0.09 mmol) of palladium acetate are added to a solution of 0.96 g (1.04 mmol) of benzyl β-D-glucopyranosyl-(1→3)-β-D-glucopyranosyl-(1→3)-β-D-glucopyranosyl-(1→3)-β-D-glucopyranosyl-(1→3)-β-D-glucopyranoside in 20 mL of a methanol/water (1:1) mixture, and the suspension is then stirred vigorously during 2.5 hours under hydrogen atmosphere at room temperature. After filtration and concentration, the product is submitted to gel permeation purification (Sephadex G-15, water as eluent) to give, after freeze-drying of the purified fractions, 658 mg of the desired β-D-glucopyranosyl-(1→3)-β-D-glucopyranosyl-(1→3)-β-D-glucopyranosyl-(1→3)-β-D-glucopyranosyl-(1→3)-β-D-glucopyranose of formula

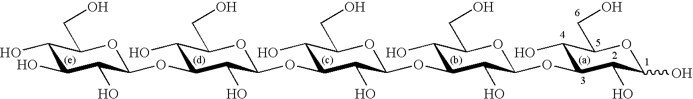

Overall yield (3 steps): 71%

TLC: R_{f}=0.2 (ethyl acetate/isopropanol/water, 3:3:2) [[α]²⁰_{D}=−9 (t=10 min, c=1,0; water)

RMN ¹H (D₂O, 400 MHz) 5 (ppm): 5.12 (d, 1H, H1aα, J_{H1aα-H2a}=3.8 Hz); 4.68 (d, 2H, H1, J_{H1-H2}=8.0 Hz); 4.68 (d, 2H, H1, J_{H1-H2}=8.1 Hz); 4.66 (d, 2H, H1, J_{H1-H2}=7.8 Hz); 4.64 (d, 2H, H1, J_{H1-H2}=7.7 Hz); 4.56 (d, 1H, H1aβ, J_{H1aβ-H2a}=8.0 Hz); 3.82-3.58 (m, 30H); 3.47-3.23 (m, 30H). MS:
[M+Na]⁺ m/z: calculated=851, 2645; found=851, 2650
[M−H+2Na]⁺ m/z: calculated=873, 2464; found=873, 2525

### Example 3

### Effect of Laminaritetraose and Laminaripentaose on Phagocytosis of Cells from Peripheral Blood

A group of Balb/c mice (Jackson laboratory, Bar Harbor, ME, USA) has been injected intraperitoneally with either PBS (Control; Sigma, St. Louis, Mo., USA), Laminaritetraose or Laminaripentaose. 24 hrs later, mice were sacrified, peripheral blood from the orbital plexus was collected into heparine (5 IU/ml) (Sigma).

After counting, a test of phagocytosis of HEMA particles (synthetic microspheres prepared from 2-hydroxyethylmethacrylate copolymer) was performed as described in: Rembaum et al., 1976, Vetvicka et al. 1982, Bilej et al., 1989.

0.1 ml of heparinized fresh blood was added to 0.05 ml of diluted HEMA particles (5×10⁸/ml) and incubated for 60 minutes at 37° C. with occasional gentle agitation.

After the end of incubation, the cell suspension was smeared over microscope slides. Smears were evaluated under the optical microscope after Accustain (modified Wright stain, Sigma) staining.

Cells with at least three engulfed particles were considered to be positive.

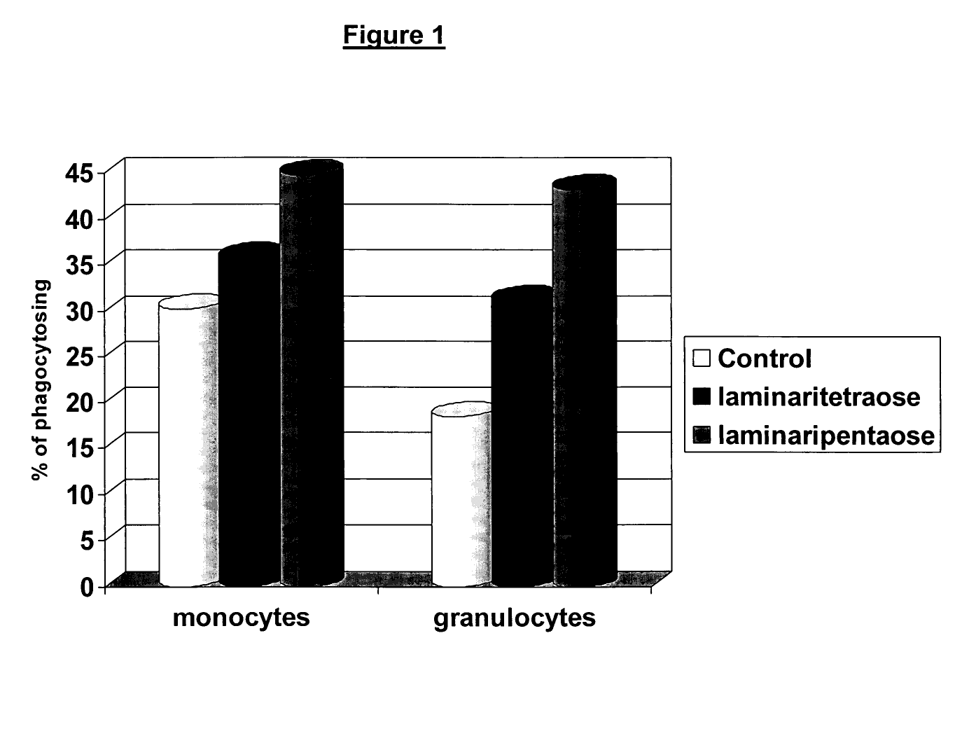

The results are given in the following Table 1 and the mean results are represented graphically in FIG. 1.

The results clearly show that both Laminaritetraose and Laminaripentaose strongly stimulate phagocytosis in both monocytes and granulocytes.

TABLE 1

| | % of phagocytosing | % of phagocytosing |
|---|---|---|
| | monocytes | granulocytes |
| Control | 30.5 | 14.6 |
| | 31.4 | 18.6 |
| | 28.7 | 21.9 |
| mean | 30.2 | 18.4 |
| Laminaritetraose | 39.8 | 32.6 |
| | 36.8 | 28.6 |
| | 30.9 | 32.3 |
| mean | 35.8 | 31.2 |
| Laminaripentaose | 41.6 | 40.8 |
| | 43.1 | 42.5 |
| | 49.6 | 46.4 |
| mean | 44.8 | 43.2 |

### Example 4

### Effect of Laminaritetraose and Laminaripentaose on Phagocytosis of Cells from Peritoneal Cavity

A group of Balb/c mice (Jackson laboratory, Bar Harbor, Me., USA) has been injected intraperitoneally with either PBS (control), Laminaritetraose or Laminaripentaose. 24 hrs later, mice were sacrificed, peritoneal cells were collected into Hanks medium (Sigma).

After counting the cells in hemocytometer, the peritoneal cells were diluted to lxlO7 cells in RPMI 1640 medium (Sigma) with 5% fetal calf serum (Hyclone, Logan, Utah, USA).

2×10⁶ cells in 0.2 ml of RPMI 1640 medium supplemented with 5% fetal calf serum were mixed with the same volume of HEMA particles (5×10⁸/ml).

The suspension was incubated for 60 minutes at 37° C. with occasional agitation. Incubation was terminated by centrifugation (150 g for 5 minutes) and the pellet was resuspended.

Macrophages with ingested particles were scored under the optical microscope in smears after Accustain (modified Wright stain, Sigma) staining.

Cells with six or more engulfed particles were considered to be positive.

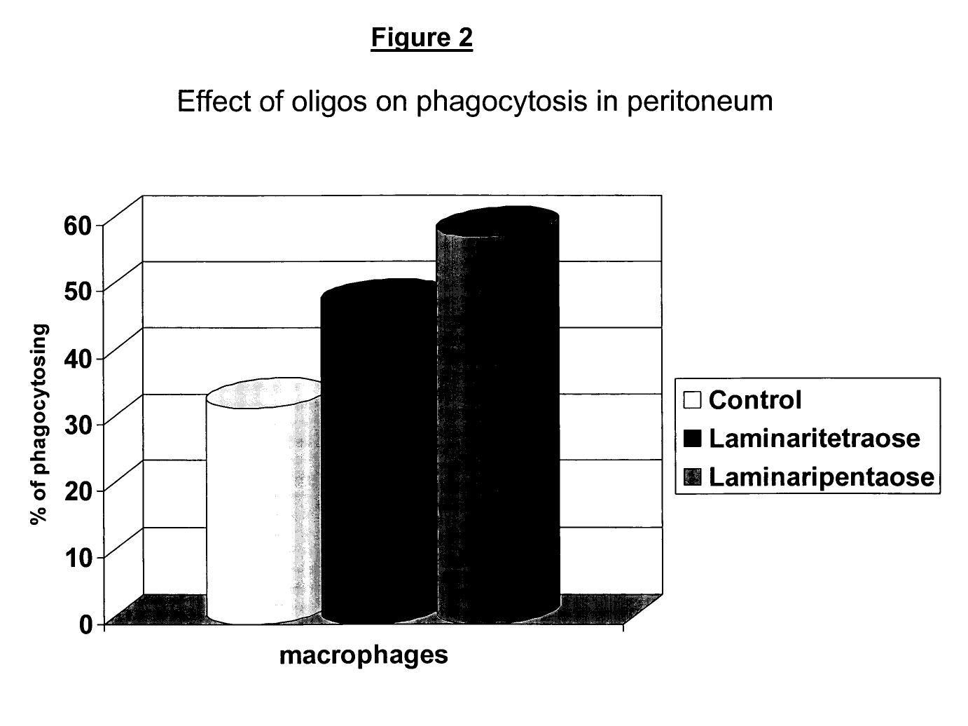

The results are given in the following Table 2 and the mean results are represented graphically in FIG. 2.

They show that both Laminaritetraose and Laminaripentaose strongly stimulate phagocytosis in both monocytes and granulocytes of peritoneal macrophages.

TABLE 2

| | % of phagocytosing |
|---|---|
| | macrophages |
| Control | 31.4 |
| | 30.9 |
| | 35.2 |
| mean | 32.5 |
| Laminaritetraose | 44.5 |
| | 46.2 |
| | 51.5 |
| mean | 47.4 |
| Laminaripentaose | 54.9 |
| | 57.3 |
| | 62.7 |
| mean | 58.3 |

### Example 5

### Effect of Laminaritetraose and Lamninaripentaose on Differential Count in Blood

Using the same experimental groups as described above in Example 3, two extra microscopic slides from each experimental sample were prepared. After Accustain (modified Wright stain, Sigma) staining, the slides were evaluated using the optical microscope for presence of individual types of cells, i.e. monocytes, lymphocytes and granulocytes.

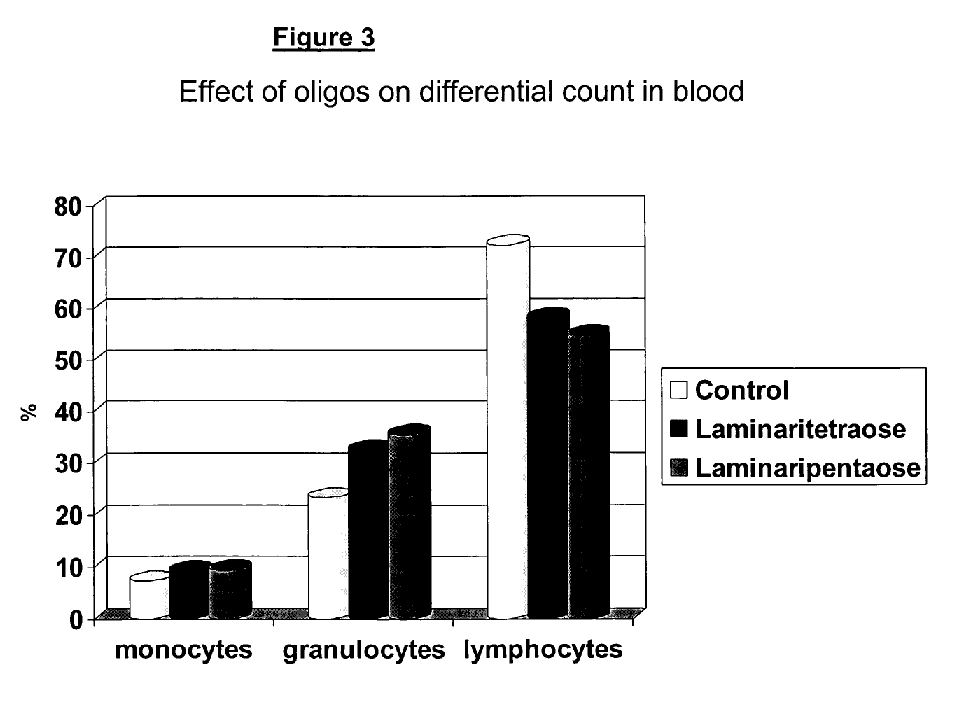

The results are given in the following Table 3 and the mean results are represented graphically in FIG. 3.

They show that both Laminaritetraose and Laminaripentaose increase the number of granulocytes in the peripheral blood.

TABLE 3

| | % of | % of | % of |
|---|---|---|---|
| | monocytes | granulocytes | lymphocytes |
| Control | 6.6 | 23.4 | 79.0 |
| | 5.6 | 25.5 | 68.9 |
| | 9.6 | 21.3 | 69.1 |
| mean | 7.3 | 23.4 | 72.3 |
| Laminaritetraose | 8.5 | 30.1 | 61.4 |
| | 9.1 | 33.2 | 57.7 |
| | 10.0 | 34.4 | 55.6 |
| mean | 9.2 | 32.6 | 58.2 |
| Laminaripentaose | 9.1 | 33.8 | 57.1 |
| | 1.0 | 35.9 | 54.1 |
| | 9.8 | 36.9 | 53.3 |
| mean | 9.4 | 35.5 | 54.8 |

### Example 6

### Effect of Laminaritetraose and Lamninaripentaose on Differential Count in Peritoneal Cells

Using the same experimental groups as described above in Example 4, two extra microscopic slides from each experimental sample were prepared. After Accustain (modified Wright stain, Sigma) staining, the slides were evaluated using the optical microscope for presence of individual types of cells, i.e. macrophages, lymphocytes and mast cells.

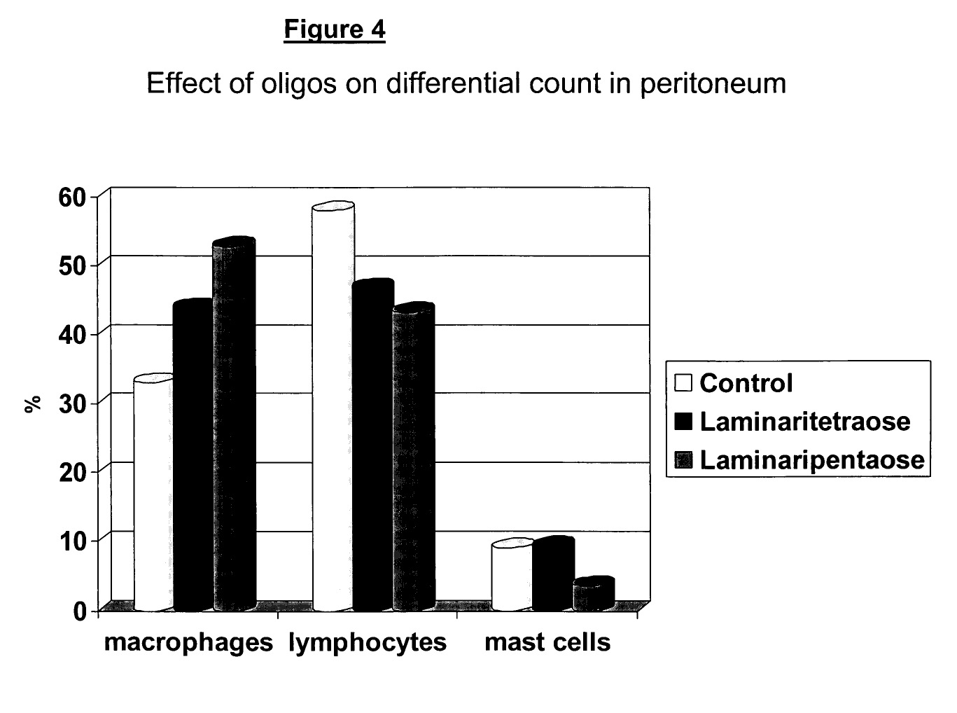

The results are given in the following Table 4 and the mean results are represented graphically in FIG. 4.

They show that both Laminaritetraose and Laminaripentaose stimulate the migration of macrophage into the peritoneal cavity.

TABLE 4

| | % of | % of | % of |
|---|---|---|---|
| | macrophages | lymphocytes | mast cells |
| Control | 35.2 | 55.9 | 8.8 |
| | 29.5 | 57.3 | 13.2 |
| | 34.1 | 60.8 | 5.1 |
| mean | 33.0 | 58.0 | 9.0 |
| Laminaritetraose | 46.8 | 44.1 | 9.1 |
| | 41.2 | 45.9 | 12.9 |
| | 43.6 | 50.4 | 6.0 |
| mean | 43.9 | 46.8 | 9.3 |
| Laminaripentaose | 50.4 | 48.1 | 1.5 |
| | 48.1 | 48.0 | 3.9 |
| | 59.8 | 34.7 | 5.5 |
| mean | 52.8 | 43.4 | 3.6 |

### Example 7

### Effect of the Laminaritetraose and Laminaripentaose on Cytokine Level in Peripherical Blood

Balb/c mice were intraperitoneally injected with 50 μg of Laminaritetraose, Laminaripentaose, Laminarin or Lentinan (purchased from NIH, Bethesda, Md., USA) in PBS.

After various time intervals (30, 60 and 90 minutes, respectively), after the injection of Laminaritetraose, Laminaripentaose, Laminarin, Lentinan only, the mice were killed and blood was collected in Eppendorf tubes.

Subsequently, the serum of the collected blood was separated, collected and stored at −80° C. for no more than 1 week.

The level of TNF-alpha in the serum samples was evaluated using a commercial kit marketed as OptEIA Mouse TNF-alpha (Mono/Mono) Set by the Company Pharmingen, San Diego, Calif., USA); the manufacturer's instructions were followed.

In connection with the experiments carried out by Applicants, Balb/c mice were intraperitoneally injected with various doses (50, 100, and 250 μg) of Soluble laminarin and Lentinan (from NIH, Bethesda, Md., USA) in PBS.

Control mice were treated with PBS only.

After various time intervals (10, 30 and 60 minutes, respectively), the mice were killed and blood was collected in Eppendorf tubes.

Subsequently, the serum was prepared, collected and stored at −80° C. for no more than 1 week.

The level of TNF-alpha in serum samples was evaluated using a commercial kit marketed as OptEIA Mouse TNF-alpha (Mono/Mono) Set by the Company Pharmingen, San Diego, Calif., USA); the manufacturer's instructions were followed.

In that respect, The wells of 96-well plates were coated with 0.1 ml/well of capture antibody (provided in the above kit) diluted in coating buffer (also provided in the above kit); the expression “capture antibody” designates first antibody used for coating of wells; this antibody captures the tested cytokines from the solution; in this assay it was anti-mouse-TNF-alpha monoclonal antibody.

The plates were sealed and incubated overnight at 4° C.

Individual wells were emptied by aspiration and washed 3 times with over 300 el/well of wash buffer (also provided in the kit).

Reaction was blocked with 200 el/well of assay dilutant diluent (also provided in the kit) and by incubation for 60 minutes at room temperature.

Again, individual wells were emptied by aspiration and washed 3 times with over 300 el/well of the same wash buffer.

Standards (also provided in the kit) and samples of serum were diluted in assay diluent (also provided in the kit) and pipetted (100 μl/well) in appropriate wells; as far as the dissolution rate is concerned standards (part of the kit) were diluted according to the instructions into following concentrations: 1000 pg/ml, 500 pg/ml, 250, 125, 62.5, 31.3, and 15.6 pg/ml.

The plates were sealed and incubated for 60 minutes at room temperature. Individual wells were aspirated and washed 3 times with over 300 μl/well of the same wash buffer. A quantity of 100 μl/well of a working detector (antibody-avidin-HRP conjugate also provided in the kit) was added into each well.

The plates were sealed with plastic foils and incubated for 60 minutes at room temperature. Individual wells were emptied by aspiration and washed 3 times with over 300 μl/well of same wash buffer.

A quantity of 100 μl/well of substrate solution (also provided in the kit) was added to each well and the plates were incubated for 30 minutes in the dark at room temperature; “substrate solution” is formed by mixing a substrate reagent A containing hydrogen peroxide and Substrate reagent B containing 3,3′,5,5′-tetramethylbenzidine in organic solvent; when mixed together, the reagent reacts with peroxidase-labeled conjugates to develop a blue color.

A quantity of 50 μl/well of stop solution provided in the kit and adapted to stop the reaction was added to each well and the optical density was determined using a STL ELISA reader (marketed by Tecan U.S., Research Triangle Park, N.C.) at 450 nm with a correction at 570 nm.

The concentration of TNF-alpha, in pg/ml, in the blood of the mice treated as hereabove disclosed has been determined at the following moments: 30, 60 and 90 minutes after the injection of Laminaritetraose, Laminaripentaose, Lentinan and control.

The values obtained are collected in Table 5.

TABLE 5
Concentration of TNF alpha in pg/ml of blood of treated mice after
different durations of treatment

| | 30 min | 60 min | 90 min |
|---|---|---|---|
| Laminaritetraose | 55.7 | 103.0 | 35.3 |
| 50 μm/mouse | | | |
| Laminaripentaose | 34.3 | 37.0 | 71.3 |
| 50 μm/mouse | | | |
| Lentinan | 20 | 15.7 | 32.0 |
| 50 μm/mouse | | | |
| Laminaritetraose | 39.0 | 18.4 | 13.6 |
| 100 μm/mouse | | | |
| Laminaripentaose | 53.0 | 46.5 | 93.2 |
| 100 μm/mouse | | | |
| Lentinan | 28 | 30.7 | 20.3 |
| 100 μm/mouse | | | |
| Laminaritetraose | 27.3 | 152.3 | 11.1 |
| 250 μm/mouse | | | |
| Laminaripentaose | 42.1 | 36.4 | 24.4 |
| 250 μm/mouse | | | |
| Lentinan | 86.3 | 68.7 | 48.6 |
| 250 μm/mouse | | | |

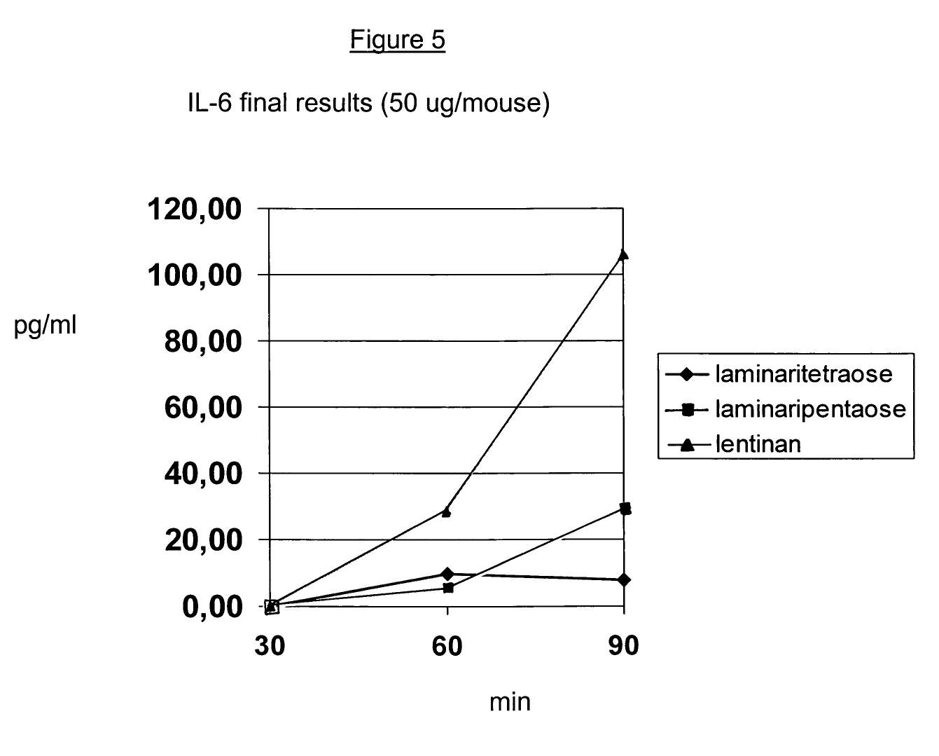

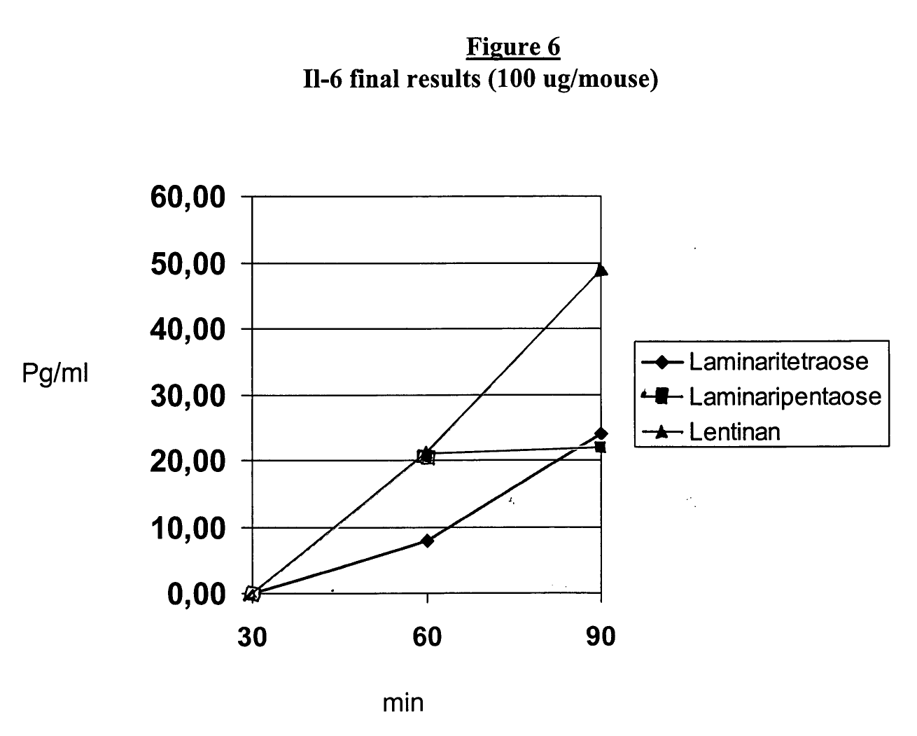

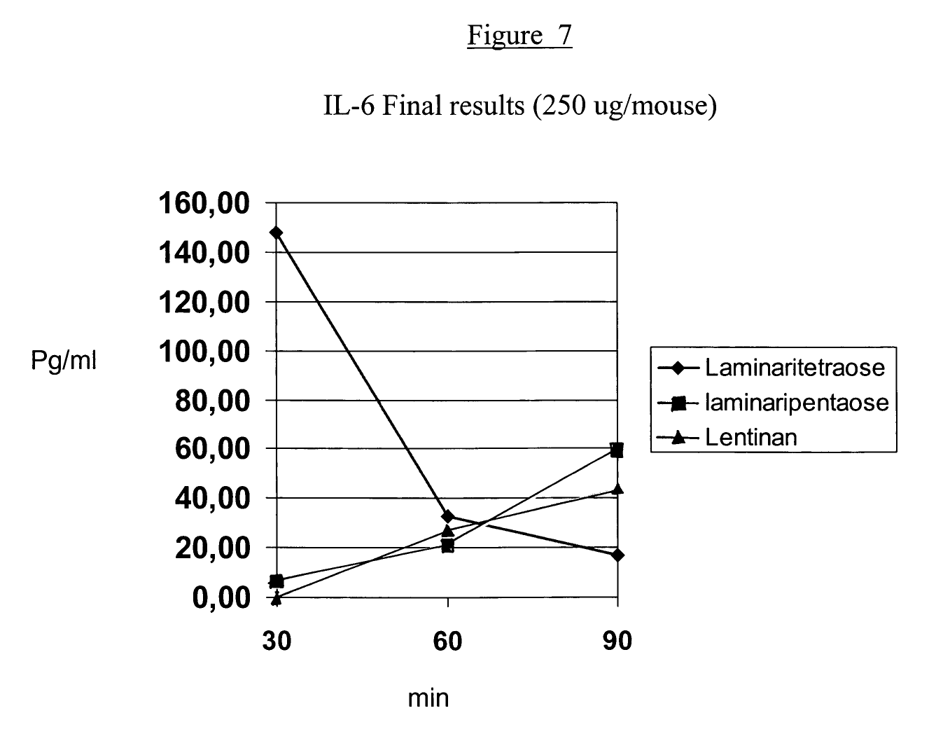

FIGS. 5, 6 and 7 are graphs representing the variation of the concentration expressed in pm/ml of TNF-alpha in the blood of the experimental mice as a function of the duration t, expressed in minutes of the action of Laminaritetraose, Laminaripentaose and Lentinan, respectively at dosage of 50 μm/mouse, 100 μm/mouse and 250 μm/mouse.

The conclusions which can be drawn from the data of table 5 and FIGS. 5 to 7, are that the indirect activation of macrophages and cytotoxic T lymphocytes, measured as the increase of TNF-alpha secretion is significantly higher when using low doses of Laminaritetraose or Laminaripentaose, instead ofLentinan.

The same experiments as those described above are carried out with 250 μm/mouse of Laminarin, Laminaritetraose and Laminaripentaose, and the measures were performed after 24 and 48 hours.

The results are given in the following table 6.

TABLE 6

| | 24 hours | 48 hours |
|---|---|---|
| Laminaritetraose | 28.1 | 35.9 |
| Laminaripentaose | 79.6 | 38.7 |
| Laminarin | 19.4 | — |

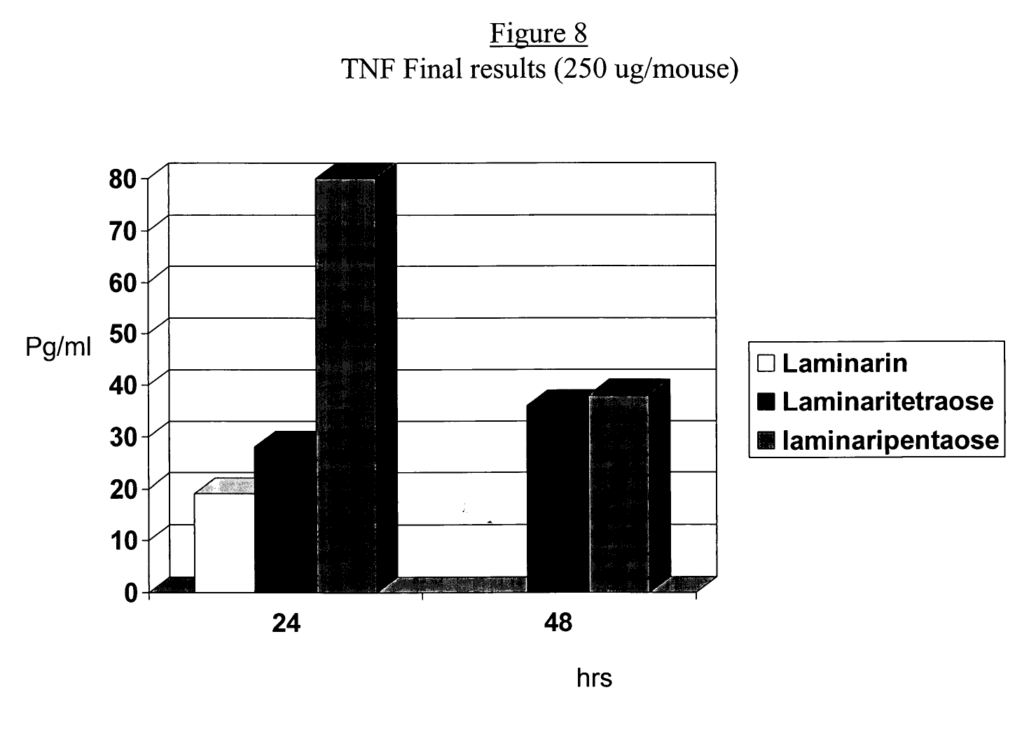

Those results are presented under graphically form on FIG. 8.

Those results show that Laminaripentaose is more active than Laminaritetraose. Both oligo-β-(1,3)-glucans are effective in large intervalle( 24 and 48h).

Laminaritetraose and laminaripentaose are more active than yest derived glucan (Lentinan).

1. A therapeutical method comprising administration of a composition comprising an amount of oligo-β-(1,3)-glucan and a pharmaceutically acceptable carrier, to a human being or to a warm-blood animal suffering from a disease selected from the group consisting in a tumor, a cancer, a viral disease, a bacterial disease, a fungal disease, a disease of the immune system, an auto-immune disease or a disease related to a deficiency of immunostimulation, wherein the amount of oligo-β-(1,3)-glucan is effective to treat the disease.

2. The method according to claim 1, wherein the oligo-O-(1,3)-glucan is a compound presenting the following formula (1): 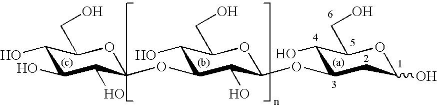
in which n=1 to 7, preferably, n=2 or n=3, or a pharmaceutically acceptable salt therof.

3. The method according to claim 2, wherein the compound of formula 1 is Laminaritetraose (n=2) or Laminaripentaose (n=3).

4. The method of claim 1, wherein the cancer is breast cancer, lung cancer, oesophagus cancer, stomach cancer, intestinal cancer or colon cancer.

5. The method according to claim 1, wherein the administration of oligo-β-(1,3)-glucan stimulates NK cells in the human being or the warm-blood animal.

6. The method according to claim 1, wherein the administration of oligo-β-(1,3)-glucan stimulates the production of TNF-alpha in the human being or the warm-blood animal.

7. The method according to claim 1, wherein the composition is administered intravenously, intraperitoneally, or orally to the patient.

8. The method according to claim 1, further comprising administration of a chemotherapeutic agent.

9. The method according to claim 1, further comprising administration of a potentiator.

10. The method according to claim 1, wherein the composition is under the form of a solution, suspension, syrup, tablet, capsule, injections, ointment, pulmonary spray.

11. Pharmaceutical composition under the form of a solution, suspension, spray, aerosol, tablet, capsule, injection, ointment, pulmonary aerosol comprising a therapeutically effective amount of oligo-1-β-O-glucan of formula (1) 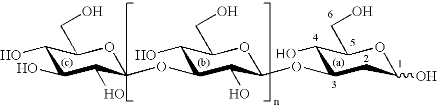
in which n=1 to 7, preferably n=2 or n=3,
or a salt pharmaceutically acceptable salt thereof,
and a pharmaceutical acceptable carrier,
said composition being free of any other glucan.
